(19) United States
(12) Patent Application Publication
(10) Pub. No.: US 2007/0232701 A1
(43) Pub. Date: Oct. 4, 2007

(54) Synergistic compositions useful as cytostatic, bacteriostatic and/or virostatic agents

(76) Inventor: Corinne E. Griguer, Birmingham, AL (US)

Correspondence Address: BIOTECH BEACH LAW GROUP , PC, 625 BROASWAY , STE. 1210, SAN DIEGO, CA, 92101, US

(21) Appl. No.: 11/703,261

(22) Filed: Feb. 7, 2007

(30) Foreign Application Priority Data
Apr. 3, 2006 (CA) 2,541,636

(51) Int. Cl. A61K 31/12

(52) U.S. Cl. 514/688; 514/678

(57) ABSTRACT

The inventor has surprisingly found that a combination of water-soluble organic compounds provides a synergistic effect that efficiently blocks ATP production by the aerobic pathway. More particularly, such a synergistic composition lowers the level of energy or production of ATP to a point where cells and specifically cancer cells, stop dividing. In this connection, the present invention provides a synergistic composition which is particularly useful as potent cytostatic and/or antibiotic agents and their use in methods for treating cancers and/or bacterial infections.

### CROSS-REFERENCE TO RELATED APPLICATIONS

The present application claims benefit of priority to Canadian provisional patent application serial number 2,541,636 filed on Apr. 3, 2006, the disclosure of which is herein incorporated by reference in its entirety.

### FIELD OF THE INVENTION

The present invention relates to novel synergistic compositions useful as potent cytostatic and/or bacteriostatic and/or virostatic agents and their use against proliferative diseases, such as cancer.

### BACKGROUND OF THE INVENTION

Metabolic systems are identical in all eukaryotic cells. They are responsible for the conversion of matter into energy of adenosine triphosphate (ATP) and therefore into the many work functions necessary for life. When these systems are impaired by defective genes or proteins or by environmental conditions, they fail to supply energy necessary for the basic cellular functions, and the cell fails as a healthy entity. These systems are also equipped with a variety of redundant mechanisms: alternative pathways through which normal level of “bioenergetic potential” may be restored and maintained even in the presence of genetic or environmental insult. Metabolic control analysis may reveal on a cell- and tissue-specific level, change in redox potential and key redox-sensing proteins that in turn are related to changes in gene expression and therefore to disease phenotype. Therefore, modification of metabolism can potentially alter cell phenotype. As biological chaos underlies the progression of tumors, enforcement of principles of metabolic control may be effective in managing cancer.

Glucose is an essential energy source for many life forms, and its deprivation can lead to growth arrest in yeast and mammalian cells. In many normal cells, glucose is catabolized to pyruvate to generate ATP and NADH. Under aerobic conditions, pyruvate is further metabolized in mitochondria to generate ATP through oxidative phosphorylation. Glucose is also required for mitogen-activated rat thymocytes to undergo proliferation that is coupled with a transition from aerobic to anaerobic metabolism, an 18-fold increase in glucose utilization, and an 8-fold induction of glycolytic enzymes. Tumor cells characteristically maintain a high glycolytic rate even under aerobic conditions, a phenomenon recognized by Warburg seven decades ago.

It is also well established that aerobic glycolysis is actively stimulated in transformed cells; in particular, glycolysis is stimulated in response to Transforming Growth Factor (TGF) β in cells transfected with c-myc oncogene. The association between transforming oncogenes and glycolysis is suggestive of a link between cell proliferation and energy metabolism. In support of this hypothesis, quiescent thymocytes cultured in absence of mitogenic growth factors derive more than 80% of their ATP from oxidative metabolism whereas the same thymocytes population subjected to mitogenic stimulation derive more than 80% of their ATP from glycolytic metabolism. Cancer cells produce more than 80% of the principal “fuel” for the cell, ATP, from glycolytic metabolism and about 20% ATP is produced by oxidative phosphorylation. It has been proposed that in spite of maintaining an accelerated glycolysis, ATP needed to support the most important cellular mechanisms, (i.e., protein synthesis, active ion transport) is produced by oxidative phosphorylation, with glycolysis playing a minor role. In glioma, many studies have described a very high rate of glycolysis. The high rate of glycolysis described in many tumors and in gliomas can be explained by up-regulation of several glycolytic enzymes.

However, there is an obvious conflict during ischemia, because both the oxygen and glucose supply are insufficient. Thus cells and tissues, such as those of tumors, are confronted by an insufficiency of both oxygen and nutrients, and the switch to glycolysis seems inadequate to explain the adaptation. Many reports have challenged the Warburg theory, in particular in thyroid oncocytoma, in hepatoma, and glioma cell lines, where oxidative energetic metabolism is observed.

Hence partial and selective block of the oxidative respiration at the level of the electron transport chain (ETC) would lower the production of ATP and therefore inhibit essential functions.

Therefore, modulator of the electron transporters chain, designed to capture the electrons flowing down the different complexes of the ETC are potential new categories of potent anti cell proliferation and anticancer compounds that can act selectively in cancer cells with no acute side effects. Thus, there is a need to provide new compositions which comprises such agents.

### SUMMARY OF THE INVENTION

The present invention fulfils these needs and other needs which will be apparent to those skilled in the art upon reading the following specification.

More specifically that object is achieved by a synergistic composition comprising:

a) a first compound consisting of a quinone derivative of the general formula A

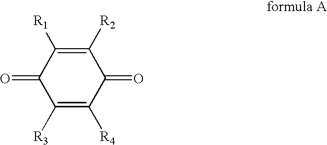

where R₁, R₂, R₃, R₄ are the same or different and are selected from the group consisting of H, OH, CH₃, OCH₃, CHO, COOH, halogen atoms (Br; Cl; F; I), NO₂ NH₂ and SO₃H;
b) a second compound selected from the group consisting of:
a 3-cyclobutene-1,2 dione derivative of the general formula B1

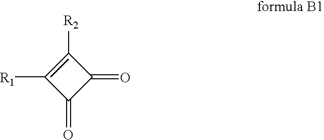

where R₁ and R₂ are the same or different and are selected from the group consisting of H, OH, CH₃, OCH₃, CHO, COOH, halogen atoms (Br; Cl; F; I), NO₂, NH₂ and SO₃H;
a 4-cyclopentene-1,2,3, trione derivative of the general formula B2

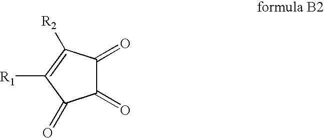

where R₁ and R₂ are the same or different and are selected from the group consisting of H, OH, CH₃, OCH₃, CHO, COOH, halogen atoms (Br; Cl; F; I), NO₂, NH₂ and SO₃H; and
a 5-cyclohexene-1,2,3,4 tetraone derivative of the general formula B3

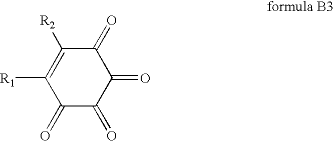

where R₁ and R₂ are the same or different and are chosen from the group consisting of H, OH, CH₃, OCH₃, CHO, COOH, halogen atoms (Br; Cl; F; I), NO₂. NH₂ and SO₃H;
   and
   c) a third compound consisting of benzene derivative of the general formula C

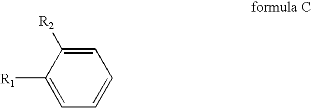

where R₁ and R₂ are the same or different and are chosen from the group consisting of H, OH, CH₃, OCH₃, CHO, COOH, halogen atoms (Br; Cl; F; I), NO₂, NH₂ and SO₃H.

Another object of the present invention is the use of the composition of the inventions as a cytostatic agent, more specifically against tumor and/or cancer cells.

Yet another object of the present invention is the use of the composition of the invention as an antibiotic agent, such as a bacteriostatic agent or a bactericidal agent, or as a virostatic agent.

In still another object of the present invention is a pharmaceutical composition including the synergistic composition and a pharmaceutically acceptable carrier.

The present invention also includes to a method for treatment of cancer, comprising the step of administering to a subject in need thereof, an effective amount of a composition according to the invention. In various embodiments, the synergistic composition of the present invention is administered in combination with one or more additional compounds. In one embodiment the present invention is administered in combination with an antagonist of mTOR, such as Rapamycin. In another embodiment, the composition of the present invention is administered in combination with a compound that blocks glycolysis, such as 2-Deoxyglucose.

The present invention also includes to a method for treating a bacterial infection in a subject, comprising the step of administering to a subject in need thereof, an effective amount of a composition as defined above.

The present invention also includes a method for treating a viral infection in a subject, comprising the step of administering to a subject in need thereof, an effective amount of a composition as provided herein.

The present invention also includes a method of inducing cytostasis in a cell, comprising administering to said cell, an effective amount of a composition according to the invention.

### BRIEF DESCRIPTION OF THE FIGURES

FIG. 1 is a diagram showing the Electron Transport Chain as known in the art in both eukaryotic and prokaryotic cells. Electrons and protons (arrows) move through the different complexes based on the redox potential of those complexes.

FIG. 2 is a diagram showing the reverse phase analytical HPLC profile of the composition of the invention (Epic Polar column). The mobile phase contained 50 mM PO₃H₄ and the elution was performed by a linear gradient of acetonitrile from 0 to 70% in water. Solvent flow, 0.5 ml/min; column eluate were monitored at 260 nm. A=THQ; B=oxocarbon acids; C=catechol

FIG. 3 is a diagram showing the antibiotic activity of the composition of the invention in Gram negative E. coli cultures (A) and Gram positive Bacillus Anthracis in vitro (B) and in mice (C). The effects of the composition of the invention on bacterial growth were examined in E. coli cultures at a concentration of 5 μg/ml. The A₆₆₀ of the cultures were determined every 60 minutes during 8 hours. The effects of the composition of the invention was tested in mouse bacillus anthracis death using the BACLIGHT Bacterial Membrane Potential Kit. For the in vivo experiment, mice were injected intraperitoneally with Bacillus anthracis Sterne spores (10⁹) and treated with vehicle (PBS) or PBS plus the composition of the invention. Mouse survival was monitored and graphed as a Kaplan Meier plots.

FIG. 4 is a diagram showing the antiproliferative activity of the composition of the invention in different cancer cell lines using Live/Dead assay.

FIG. 5A-C show the antiproliferative activity of the composition of the invention in human glioma cell lines. FIG. 5A represents the percentage of live glioma cells (black bars) and dead cells (white bars) quantified by vital staining as the function of the composition of the invention concentrations when applied for 24 hours. FIG. 5B shows the total number of live cells (black bars) and dead cells (white bars) quantified by trypan exclusion method as the function of the composition of the invention concentrations for 24 consecutive days. FIG. 5C shows an average distribution of cell in G1, S and G2/M phases using flow cytometry analysis of the cell cycle in gliomas cells in presence of the composition of the invention (1 μg/ml) during 30 hours (black bars) and control condition (white bars).

FIGS. 6A-D show the survival curves in four different intracranial mouse model of gliomas. Glioma cells (0.5×10⁶/10 μl) (FIG. 6A: D-54MG; FIG. 6B: GL261; FIG. 6C: D-54MG in combination with radiation; FIG. 6D: U251 MG in combination with radiation) were used to establish intracranial gliomas in female scid mice. After 7 days, the mice were randomized to 4 groups and began a 3 week regimen of intraperitoneal injections of saline, or one of 3 doses of CEG-002 30, 60 and 120 mg/ml respectively. For the radiation modality, mice were treated with 30 mg/ml of CEG-002 5 days prior the first radiation.

### DETAILED DESCRIPTION OF THE INVENTION

The inventor has surprisingly found that the combination of water-soluble organic compounds provides a synergistic effect that efficiently blocks ATP production by the aerobic pathway. More particularly, such a synergistic composition lowers the level of energy or production of ATP to a point where cells and specifically cancer cells, stop dividing. In this connection, the present invention provides a synergistic composition which is particularly useful as potent cytostatic and/or antibiotic agents and their use in methods for treating cancers and/or bacterial infections.

### 1. Compositions of the Invention

In a first object, the present invention relates to a synergistic composition of organic compounds which comprises a first compound consisting of a quinone derivative of the general formula A

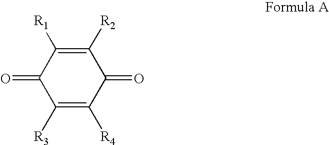

where R₁, R₂, R₃, R₄ are the same or different and are selected from the group consisting of H, OH, CH₃, OCH₃, CHO, COOH, halogen atoms (Br; Cl; F; I), NO₂, NH₂ and SO₃H.

According to a preferred embodiment, the first compound consists of tetrahydroxy-p-benzoquinone of the following formula:

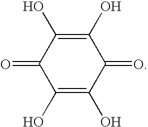

The synergistic composition of the invention also comprises a second compound selected from the group consisting of:
a 3-cyclobutene-1,2 dione derivative of the general formula B1

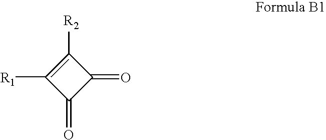

where R₁ and R₂ are the same or different and are selected from the group consisting of H, OH, CH₃, OCH₃, CHO, COOH, halogen atoms (Br; Cl; F; I), NO₂, NH₂ and SO₃H;
a 4-cyclopentene-1,2,3, trione derivative of the general formula B2

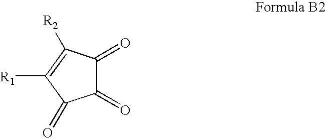

where R₁ and R₂ are the same or different and are selected from the group consisting of H, OH, CH₃. OCH₃, CHO, COOH, halogen atoms (Br; Cl; F; I), NO₂, NH₂ and SO₃H; and
a 5-cyclohexene-1,2,3,4 tetraone derivative of the general formula B3

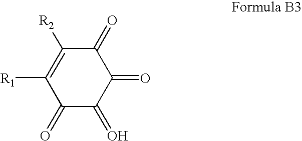

where R₁ and R₂ are the same or different and are chosen from the group consisting of H, OH, CH₃, OCH₃, CHO, COOH, halogen atoms (Br; Cl; F; I), NO₂, NH₂ and SO₃H.

The synergistic composition of the invention further comprises a third compound that consists of a benzene derivative of the general formula C

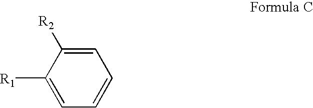

where R₁ and R₂ are the same or different and are selected from the group consisting of H, OH, CH₃, OCH₃, CHO, COOH, halogen atoms (Br; Cl; F; I), NO₂, NH₂ and SO₃H.

According to a preferred embodiment, the benzene derivative of the general formula C consists of the catechol of formula:

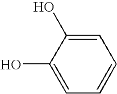

A preferred 3-cyclobutene-1,2 dione derivative of the general formula B1 contemplated by the present invention consists of squaric acid of the following formula:

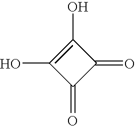

A preferred 4-cyclopentene-1,2,3, trione derivative of the general formula B2 consists of croconic acid of the following formula:

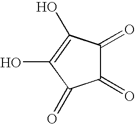

A preferred 5-cyclohexene-1,2,3,4 tetraone derivative of the general formula B3 consists of 5,6 dihydroxy-5-cyclohexene-1,2,3,4 tetraone of the following formula:

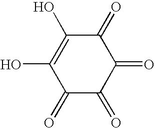

According to a preferred embodiment, the first, second and third compounds are combined following a ratio of about 3:1:1, respectively.

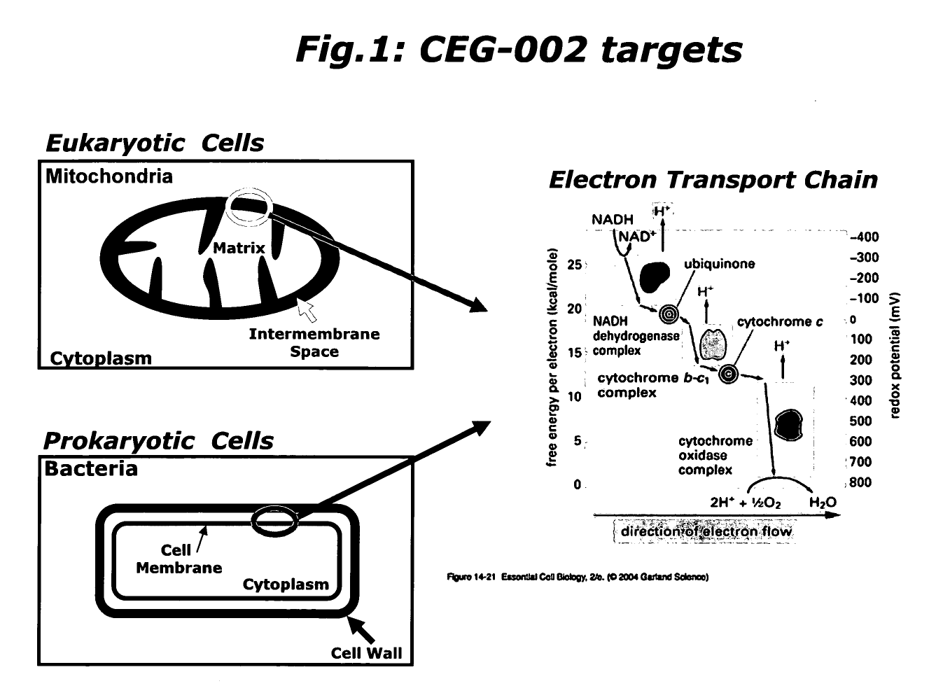

As one skilled in the art may appreciate, the composition of the invention acts as a synthetic electron carrier molecule that moves electrons from more negative redox potential to more positive ones (FIG. 1). The composition of the invention reduces the number of free electrons in the mitochondrial cytochrome system and prevents the upward flow of any electrons remaining in the system, thereby reducing ATP formation. This might be done by shunting electrons directly to oxygen using preferred redox active compounds such as those described above, which are advantageously the different electron acceptor of the electron transport chain (ETC).

The synergistic composition of the invention may further comprise an acceptable carrier. As used herein, the expression “an acceptable carrier” means a vehicle for containing the compounds of the composition of the invention that can be administered to a host without adverse effects. Suitable carriers known in the art include, but are not limited to, gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

Further agents can be added to the composition of the invention. For instance, the composition of the invention may also comprise agents such as drugs, immunostimulants (such as α-interferon, β-interferon, γ-interferon, granulocyte macrophage colony stimulator factor (GM-CSF), macrophage colony stimulator factor (M-CSF), interleukin 2 (IL2), interleukin 12 (IL12), and CpG oligonucleotides), antioxidants, surfactants, flavoring agents, volatile oils, buffering agents, dispersants, propellants, and preservatives. For preparing such compositions, methods well known in the art may be used.

### 2. Methods of Use

The synergistic compounds of the invention may be used in many ways, for instance as a cytostatic and/or a antibiotic agent, and/or a virostatic agent. It will be understood that a cytostatic agent, a antibiotic agent and a virostatic agent refers to an agent that suppresses the growth and multiplication of a cell, a bacterium, and a virus respectively.

As used herein, the term “cell” is used broadly, and includes cells in vitro, in vivo, prokaryotic, eukaryotic, and fungal to be used in accordance with the present invention. Preferred cells contemplated by the present invention are tumour and cancer cells. Even more preferably, the cancer cells are cells from a cancer selected from, but not limited to, the group consisting of carcinoma such as bladder, breast, colon, kidney, liver, lung, esophagus, gall-bladder, ovary, pancreas, stomach, cervix, thyroid, prostate, skin including squamous cell carcinoma, hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xenoderoma pigmentosum, keratoctanthoma, thyroid follicular cancer and Kaposi's sarcoma. According to another embodiment of the invention, compounds of the invention are directed to therapies for cell proliferative disorders, for example, Alzheimer's disease, viral infections, auto-immune diseases and neurodegenerative disorders.

As demonstrated in the Example section, the composition of the invention targets the inner mitochondrial potential by lowering it, therefore, as such it can be defined as a modulator of Δψm. It is thus an object of the invention to use the synergistic composition of the invention as a cytostatic agent. As one skilled in the art may appreciate, none of the compounds as defined above produce individually a cytostatic effect as one obtained by the composition of the invention. They form a supramolecular association that is highly directional and reproducible. The resulting supramolecular composition possesses unique properties that depend on cooperative interactions among the components, so that the assembly is more than just a collection of independent units. These involve a variety of possible intermodular noncovalent interactions, such as hydrogen bonding, polar attractions, van der Waals forces, and hydrophilic-hydrophobic interactions. The complex between the three components is determined in the context of the present invention by using the analytical methods described in the Example section.

It will be understood that, in the case where the composition of the invention is used to treat tumour cells in an animal, it is within the scope of the present invention to use radiation or chemotherapeutic agents. Such agents will not be described further since they are well known to one skilled in the art.

In related aspects, the present invention provides a method for treatment of cancer, comprising the step of administering to a subject in need thereof, an effective amount of the composition according to the invention.

It is also an object of the invention to use the synergistic composition as defined above as a bacteriostatic agent or a virostatic agent since it has been shown in the Example section that the composition of the invention slows down the growth rate of bacterium, for instance E. coli, kills Bacillus anthracis, and eliminates the symptoms of an FeLV (Feline Leukemia Virus) infection.

According to preferred embodiments, the synergistic composition of the invention is used in vivo or in vitro. Preferably the composition of the invention is used for treatment of bacterial or viral infection in a subject. In a related aspect, the present invention provides a method for treating a bacterial infection or a viral infection in a subject, comprising the step of administering to a subject in need thereof, an effective amount of a composition as defined above.

As used herein, the term “treating” refers to a process by which the symptoms of a disease are alleviated or completely eliminated.

An “effective amount” is an amount sufficient to effect beneficial or desired results, including clinical results, without causing overly negative effects in the host to which the composition is administered. An effective amount can be administered in one or more administrations. For purposes of this invention, an effective amount of a synergistic composition as defined above is an amount that is sufficient to palliate, ameliorate, stabilize, reverse, slow or delay the progression of the disease state. The exact amount of compounds to be used and the composition to be administered will vary according to factors such as the type of condition being treated, the mode of administration, as well as the other ingredients in the composition.

The composition of the invention described herein may be administered as a single active drug or a mixture thereof with other anti-cancer compounds, and other cancer or tumor growth inhibiting compounds or therapies such as radiation treatment or chemotherapeutic compounds. The compositions of the present invention may be provided in the form of a pharmaceutical, including the desired composition and a pharmaceutically acceptable carrier. The compounds may be administered in oral dosage forms that include tablets, capsules, pills, powders, granules, elixirs, tinctures, suspensions, syrups, and emulsions. Further, the compounds may be administered in intravenous (bolus or infusion), intraperitoneal, subcutaneous, or intramuscular form.

The composition of the invention described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used.

Techniques and compositions for making dosage forms useful for materials and methods described herein are described, for example, in the following references, which are herein incorporated by reference: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Suitable binders, lubricants, disintegrating agents, coloring agents, flavoring agents, flow-inducing agents, and melting agents may be included as carriers, e.g., for pills. For instance, an active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like.

Suitable binders include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, for example, starch, methyl cellulose, agar, bentonite, xanthan gum, and the like.

The composition of the invention may also be used with liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The composition of the invention may also be coupled to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

The composition of the invention can be administered orally in solid dosage forms, such as capsules, tablets, and powders, or in liquid dosage forms, such as elixirs, syrups, and suspensions. The active compounds can also be administered parenterally, in sterile liquid dosage forms.

Capsules may contain the active compound and powdered carriers, such as lactose, starch, cellulose derivatives, magnesium stearate, stearic acid, and the like. Similarly, such diluents can be used to make compressed tablets. Both tablets and capsules can be manufactured as immediate release products or as sustained release products to provide for continuous or long-term release of the active compounds. The deliverable form of the compounds can be sugar coated or film coated to mask any unpleasant taste and protect the tablet from the atmosphere, or enteric coated for selective disintegration in the gastrointestinal tract.

For oral administration as a liquid, the drug components may be combined with any oral, non-toxic, pharmaceutically acceptable inert carrier such as ethanol, glycerol, water, and the like. Examples liquid forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Liquid dosage forms may contain, for example, suitable solvents, preservatives, emulsifying agents, suspending agents, diluents, sweeteners, thickeners, and melting agents.

Liquid dosage forms for oral administration can contain coloring and flavoring, as needed. In general, water, suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compound of the invention described herein may also be administered in intranasal form via use of suitable intranasal vehicles, or via transdermal routes, using those forms of transdermal skin patches known to those skilled in these arts. To be administered in the form of a transdermal delivery system, the dosage administration will generally be continuous rather than intermittent throughout the dosage regimen. Parenteral and intravenous forms may also include minerals and other materials to make them compatible with the type of injection or delivery system chosen.

As used herein, the term “subject” is intended to include living organisms in which certain conditions as described herein can occur. Examples include humans, mammals and any other animals. In a preferred embodiment, the subject is a human.

The present invention further provides a method of inducing cytostasis in a cell, comprising administering to the cell, an effective amount of the composition according to the invention.

The present invention will be more readily understood by referring to the following examples. The examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the preferred methods and materials are described.

### EXAMPLES

The inventor has found that a synergistic composition according to a preferred embodiment of the invention (CEG-002) has cytostatic effects on a broad range of tumor cells in vitro and prolongs survival of severe combined immuno-deficient (scid) mice bearing intracranial brain cancer in vivo. Moreover, the composition of the invention exerts cytostatic, anti-proliferative activity by accumulating glioma cells in the S-phase of the cell cycle. Normal non-tumoral cells as well as neurons are not affected by the composition of the invention.

The following experimental procedures and materials were used for the examples set forth below.

### Materials and Methods

1. Chemicals Used in the Composition of the Invention

The preferred composition of the invention is the product of the interactions between tetrahydroxy-p-benzoquinone, squaric acid or croconic acid or 5,6 dihydroxy-5-cyclohexene-1,2,3,4 tetraone, and catechol. All were obtained from Sigma (St. Louis, Mo.).

There are three main rationales for the choice these compounds:
1- they have very similar chemical structures to elements of the electron transporter chain (ETC);
2- they can easily be oxidized or reduced to match the redox properties of the ETC complexes and therefore be donors or acceptors of electrons generated during the production of cellular energy (ATP); and
3- they are soluble in water and therefore easily cross cellular membrane (plasmatic and mitochondrial).

2. Analytical Method for Identification of the Composition of the Invention:

The process is performed by liquid chromatography (HPLC).

### Solutions Examined:

A=stock solution: 1 mg of a quinone derivative of general formula A are dissolved in mobile phase and made up to 1 ml with the same solvent.

B=Oxocarbon acids (general formula (CₙOₙ)⁻² stock solutions: 1 mg of OCA are dissolved in mobile phase and made up to 1 ml with the same solvent. Examples of oxocarbon acids utilized: 4,5-dihydroxy-4-cyclopentene-1,2,3-trione of formula B2.1 (C₅H₂O₅), or 5,6-dihydroxy-5-cyclohexene-1,2,3,4-tetraone of formula B3.1 (C₆H₂O₆).

C=Catechol stock solution: 1 mg of catechol of formula C1 is dissolved in mobile phase and made up to 1 ml with the same solvent.

The composition of the invention (also referred to as CEG-002): 1 ml of A+1 ml of B+3 ml of C each stock solution corresponding to the following concentrations: 0.2 mg/ml of A; 0.2 mg/ml of B and 0.6 mg/ml of C. Redox potential of the mixture was measured with a redox electrode with value between −400 and +800 mV.

Only the combination of the 3 compounds affords a significant cytostatic activity. The inventor has then characterized the active component using analytical HPLC.

The chromatography was carried out using two different protocols:

Protocol 1: Discovery HS C18 HPLC Column (stainless steel column of length 250 mm and inside diameter 4.6 mm), with a matrix particle platform of silica gel (particle size 5 μm) and a pre-column having the same characteristics. As mobile phase, a mixture of 20% (v/v) CH₃CN/H₂O with an isocratic elution, at a flow rate of 0.5 ml/min. With a detector set at 260 nm, 10 μl of each sample (A, B and C) was injected separately, at least three times. With regard to the separation methods used, isocratic elution was able to provide a different retention time for the 3 different compounds. Retention and quantification reproducibility were evaluated with repeated analysis and were found to be satisfactory.

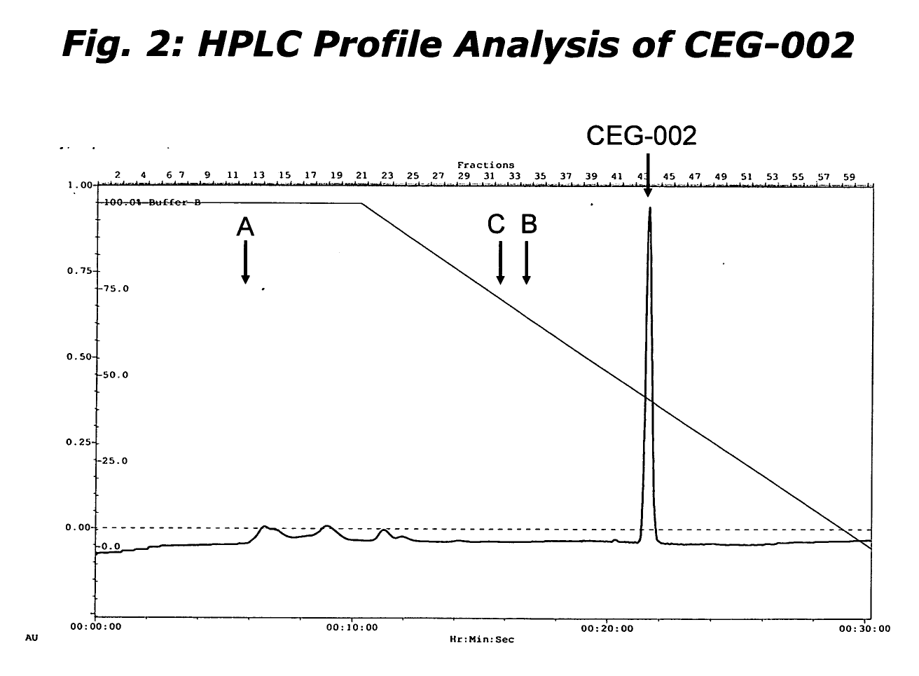

Protocol 2: The active compound from the first chromatography was collected in 1.5 ml vials and dried under reduced pressure in a vacuum centrifuge (Speed-Vac). The residue was then dissolved in mobile phase and applied for HPLC analysis in an EPIC POLAR HPLC Column (stainless steel column of length 250 mm and inside diameter 4.6 mm). As mobile phase, 50 mM PO₄H₃ and elution with a gradient of 0 to 70% (v/v) CH₃CN/H₂O, at a flow rate of 0.5 ml/min. With a detector set at 260 nm, 10 μl of each sample was injected separately, at least three times. Retention and quantification reproducibility were evaluated with repeated analysis and were found to be satisfactory. Efforts were made to analyze different combinations of the 3 compounds. As can be seen from the data provided in FIG. 2, the retention time of the composition of the invention is not consistent with the retention times previously determined for each individual component:

| Compound | Retention time (min) |
|---|---|
| A | 5.8 |
| B | 16.5 |
| C | 15.7 |
| the composition of the invention | 21.8 |

The inventor also tested the combination of the different compounds by pairs (A+C; A+B; C+B) but in any of these combinations the inventor was not able to identify the component of the composition of the invention that appears when the 3 compounds are combined together. Moreover, the pair combinations yield chromatograms with retention times corresponding to each individual compound.

### Example 1

### The Composition of the Invention is an Antibiotic for Gram Negative and Gram Positive Bacteria

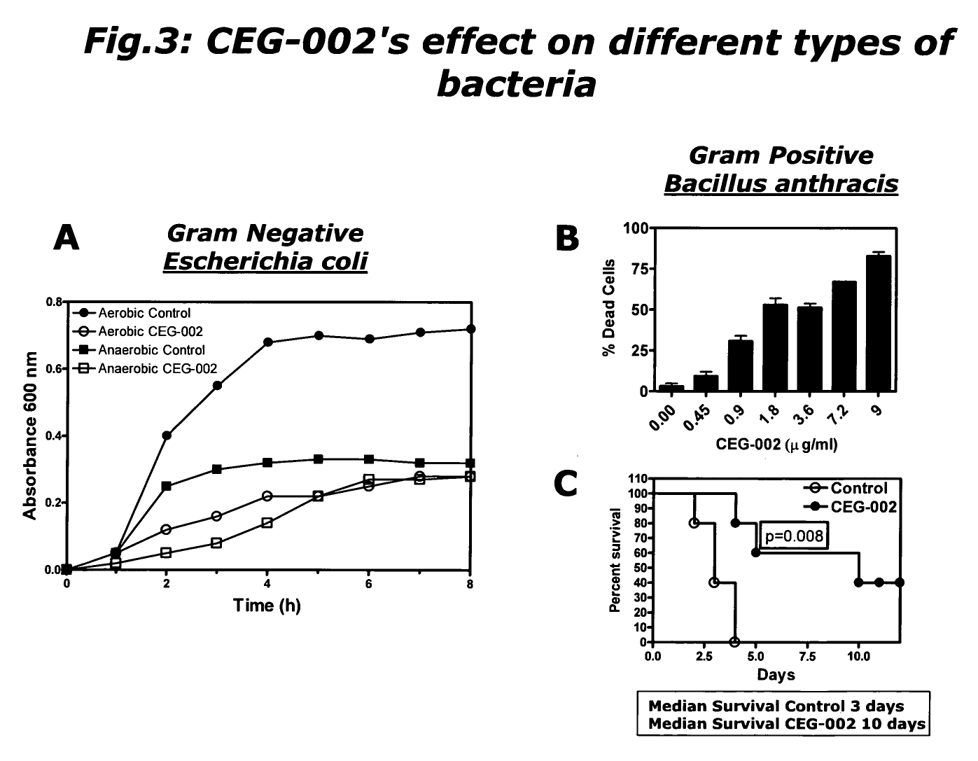

An E. coli colony from a fresh agar-plate was inoculated in 5 ml of LB medium (Fluka) and incubated in orbital shaker incubator at 37° C. over night. 0.1 mL of stationary overnight culture was inoculated into 4.5 mL LB medium and the resulting A₆₆₀ of the inoculated cultures were determined (time zero reading). The composition of the invention (5 μg/ml) was added to the culture tubes and each sample was incubated at 37° C. in a shaker bath (aerobic culture) or without shaking (anaerobic culture). The A₆₆₀ of the cultures were determined every 60 minutes during 8 hours. When the composition of the invention was applied at a concentration of 5 μg/ml, E. coli grew at a slower rate than the control culture under aerobic conditions, reaching a plateau at 6 h of culture. This result indicates that bacterial growth under aerobic condition can be reduced by 60% with the composition of the invention. An experiment was performed to determine if the effect of the composition of the invention on E. coli was bactericidal or bacteriostatic under aerobic conditions. An overnight culture was diluted and inoculated into LB medium, with or without the composition of the invention. Cell viability was monitored at intervals over a 6-h period. Then, the cells were collected and fresh medium without the composition of the invention was added, and the growth rates in control and pre-treated cultures were determined. As shown in FIG. 3, no differences in growth rates were determined, indicating that the effect of the composition of the invention is an antibiotic agent against Gram negative bacteria.

To determine the effect of the composition of the invention on a gram positive, we looked at the effect of the composition of the invention on B. anthracis, Five hour cultures of bacteria were supplemented with increasing concentrations of the composition of the invention, and bacterial cell death was measured by flow cytometry using the BACLIGHT Bacterial Membrane Potential Kit. The fluorescent membrane-potential indicator dye, DiOC₂ (3), at low concentrations exhibits green fluorescence in all bacterial cells, but it becomes more concentrated in live bacteria that are maintaining a membrane potential causing the dye to self-associate and the fluorescence emission to shift to red. Bacterial cell death was induced in a dose dependent manner with a concomitant increase in cell debris indicating a loss of viable bacteria (FIG. 3B), with a LD50 of 0.9236 μg/ml.

In order to test the composition of the invention in a mouse model, we injected 10⁹ Sterne spores intra-peritoneally in ten C57Black/6 mice. After 5 hours, the mice were randomized in two groups. The control group received intraperitoneal injection of saline or CEG-002 group 10 mg/kg every day for 3 days and survival of the mice was monitored. The median survival for the control group was 3 days whereas the CEG-002 group has a median survival of 10 days, with some mice in complete remission (FIG. 3C).

### Example 2

### The Composition of the Invention Inhibits Cell Proliferation In Vitro in Cancer Cell Lines

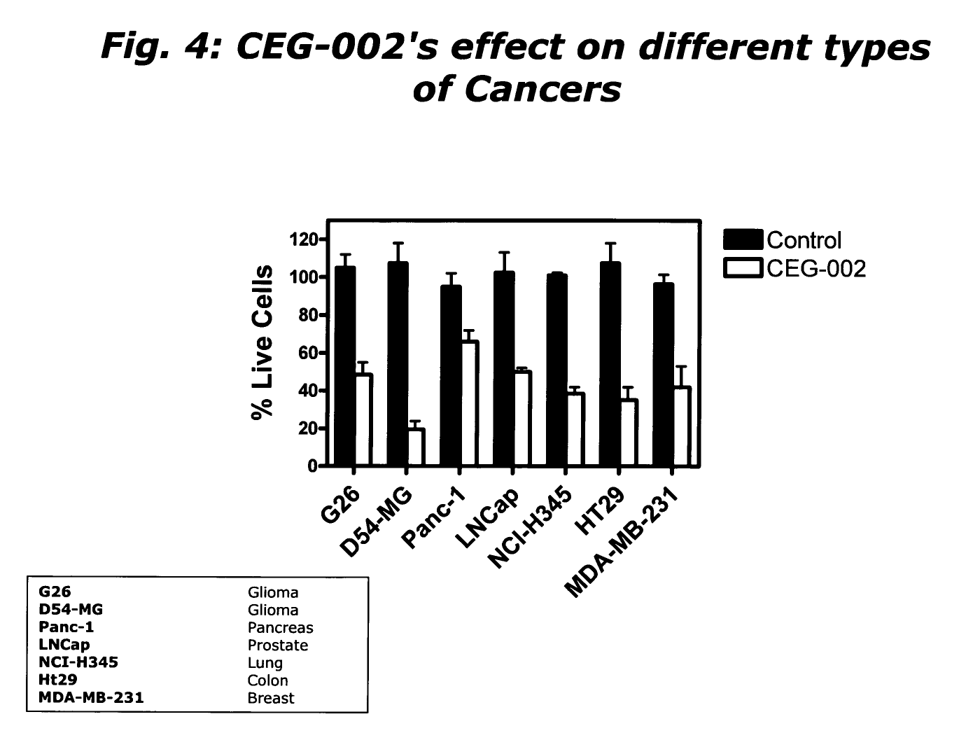

The effects of the composition of the invention on proliferation were examined in five glioma cell lines and in two adenocarcinoma cell lines using ALAMAR BLUE assay. The absorbance for cells treated with the composition of the invention was expressed as percentage to that of control cells. The cells were treated with 1 μg/ml of the composition of the invention during 48 h. It was found that D54MG, G26, GL261 (human and mouse glioma cells, Panc-1 (human carcinoma of the exocrine pancreas), LNCap (prostate cancer), NCI-H345 (small cell lung carcinoma), HT29 (colon carcinoma) and MDA-MB-231 (breast adenocarconoma), cells are sensitive to the composition of the invention. The reduction in % of live cells is related to the cytostatic activity of the composition of the invention and not to cell death. In FIG. 4, it is shown that the combination induced a decrease of the green fluorescence indicating that most of the cells under these conditions are not alive. As also seen in FIG. 4, incubation with 1 μg/ml of the composition of the invention for 48 h significantly inhibited the proliferation of all the cancer cell lines tested.

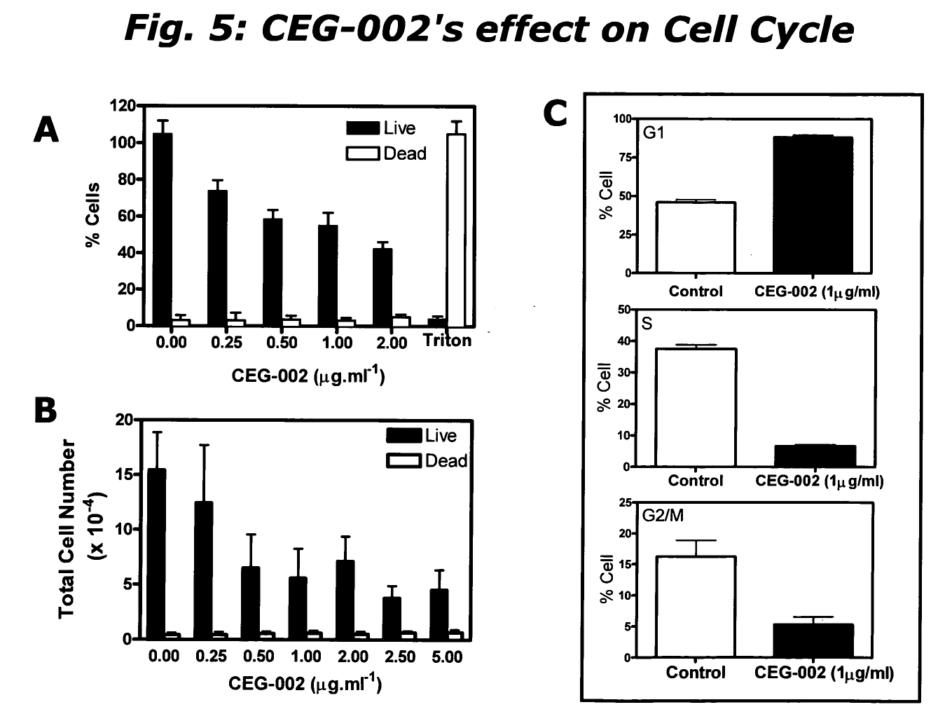

To determine cell viability, the inventor used a simultaneous vital staining by calcein AM (CAM) and ethidium homodimer-1 (EtH-1). Ethidium bromide enters cells displaying a damaged membrane and stains the nuclei of either necrotic or advanced apoptotic cells. Confluent D54 glioma cells were exposed to different doses of the composition of the invention for 24 hr. Cell viability was assessed by the ability of the cells to diesterify calcein-AM and retain the green fluorescent dye formed in the cytoplasm. Cells were incubated simultaneously with the two dyes for 10 min at 37 C, and the fluorescence was quantified with a fluorescent microplate reader (calcein, excitation 495 nm; emission 515 nm; ethidium homodimer, excitation 528 nm; emission 617 nm) (FIG. 5A). treatment with the composition of the invention produces the antiproliferative effect without cell death. This result was confirmed by trypan exclusion method (FIG. 5B). According to the results shown in FIG. 5, there is a clear reduction in the total cell number after treatment. However, no red fluorescence could be detected, indicating that activity of the composition of the invention is not related to a cell-killing effect, at least within the time frame of the experiments. These results were positively correlated with the drop in cell number, as assessed by counting the cells using trypan blue exclusion method. Together, these results indicate a significant decrease in the total number of cells with no increase in the number of cells dying. The composition of the invention induced its effects on cancer cell lines in a concentration and time-dependent manner, suggesting a specific mechanism by which the composition of the invention affects the viability of cancer cells.

### Example 4

### The Composition of the Invention Blocked Cancer Cell Cell Cycle with Selectivity

The composition of the invention inhibits cell growth without significant cell death and may act as a cytostatic agent. The cell cycle progression of cancer cells was examined using flow cytometry after exposure to the composition of the invention for 30 h. Glioma cells were synchronized for 48 h in serum free media. After 48 h, serum was reintroduced in the media with or without the composition of the invention. Decreased cell proliferation of glioma cells after treatment the composition of the invention corresponded to a significant increase in the percentage of cells in G1/S from 46±3% to 88±3%. This was accompanied by a decrease in the cells in S and G2/M of the cell cycle (FIG. 5C).

In the next set of experiments, the inventor tested the effect of the composition of the invention in normal human lung fibroblasts (CRL 1491) and mouse neurons. The cell cycle distribution did not changed significantly in presence of the composition of the invention. This result is very important because it strongly suggests that the composition of the invention is highly selective to cancer cells and can selectively synchronize cancer cells in S phase without allowing them to enter into mitosis. This effect is not present in normal senescent cells, which indicate that the composition of the invention has no toxic effect in normal cells and neurons. This is of major importance since most anti-cancer agents are highly cytotoxic for neurons and therefore induce major neurological side effects. The results indicate that the composition of the invention doesn't evoke any cytotoxicty in neurons, suggesting that the composition of the invention doesn't significantly affect non cycling cells.

### Example 5

### In Vivo Anticancer Effect of the Composition of the Invention in Intracranial Gliomas Xenograft Mouse Models

In the next set of experiments, the inventor assessed the efficacy of the composition of the invention as an anti-cancer compound. A series of experiments were performed to establish the effects of the composition of the invention in vivo in two different glioma mouse models (D-54MG, and GL261). D54MG or GL261 glioma cells (0.5×10⁶/10 ul) were used to establish intracranial gliomas in female scid mice or black 6 mice.

After 7 to 14 days depending on the model, the mice were randomized to different groups and began a 3 week regimen of intraperitoneal injections of saline, or the composition of the invention. Dosage and schedule were the same for the two models. Mice were treated with the composition of the invention intraperitoneally every day for the first five days and every other day for the last two weeks. The composition of the invention was administrated at doses between 6 to 120 mg/kg.

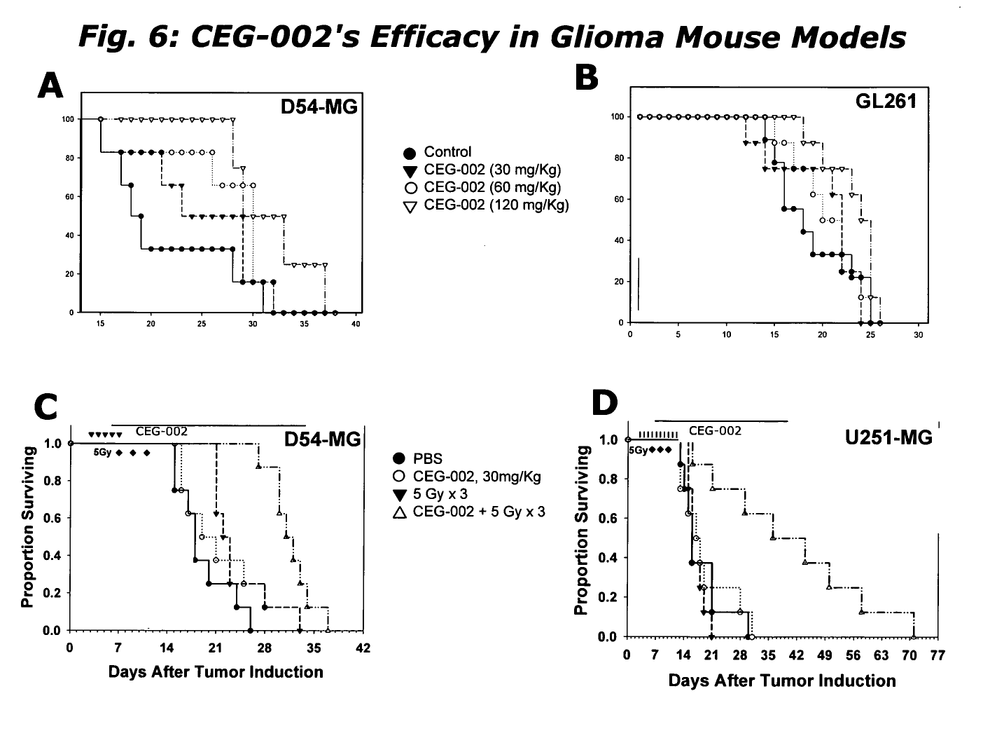

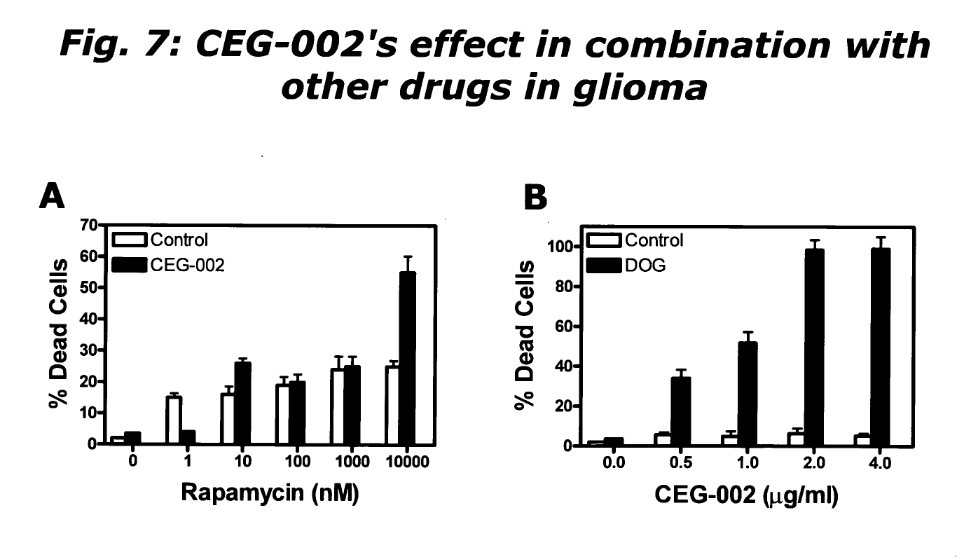

FIGS. 6 A and B illustrates survival curves for the two models. Saline treated mice had a median survival of 18 days for D-54MG (standard for this model). In contrast, the mean survival for the mice treated with the composition of the invention was 30 days, which was statistically significantly longer than the mean survival in control mice (FIG. 6 A). Survival curve for the GL261 mouse model also showed a significant increase of the median survival from 17 days for the saline treated groups to 24 days for mice treated with the composition of the invention (FIG. 6 B).

The surviving mice treated with the composition of the invention appeared to be healthy and without clinical symptoms usually associated with intracranial gliomas. Moreover, there is no significant difference in body weight between control and mice treated with the composition of the invention.

### Example 6

### The Synergistic Effect of the Composition of the Invention with Radiotherapy in Mouse Glioma Model

This set of experiments was performed in intracranial glioma mouse models where human glioma cell lines were injected intracranially in nude mice. The protocol was as follows: two weeks after induction of the tumor, the mice were randomized in four groups, mice treated with saline, mice treated with radiation alone (15 Gy, 5 Gy per week for 3 weeks), mice treated with the composition of the invention only (every other day for 3 weeks) and mice treated with both the composition of the invention and radiation (15 Gy). As shown in FIGS. 6C and 6D, there was a significant (p<0.006) increase in median survival for mice treated with both the composition of the invention and radiation. Control mice have a median survival of 17 days, median survival with radiation alone was respectively 22 and 17 days (FIGS. 6C and 6D), median survival with the composition of the invention in combination with radiation was 31 and 36 respectively corresponding to 177% and 211% increase median survival. Thus, the composition of the invention can improve survival outcome in cancer patients when combined with radiotherapy.

### Example 7

### Antagonist of mTOR such as Rapamycin or Glucose Antimetabolite 2-Deoxy-Glucose in Combination with the Composition of the Invention Produced Cytotoxicity in Cancer Cells In Vitro (FIGS. 7A and B)

Mammalian target of rapamycin (mTOR) is a serine-threonine kinase member of the cellular phosphatidylinositol 3-kinase (PI3K) pathway, which is involved in multiple biologic functions such as transcriptional and translational control. mTOR is a downstream mediator in the PI3K/Akt signaling pathway and plays a critical role in cell survival. Rapamycin is a specific mTOR antagonist that targets this pathway and blocks the downstream signaling elements, resulting in cell cycle arrest in the G₁ phase. FIG. 7A shows the effects of Rapamycin (RAPA) alone (dose response from 1 nM to 10000 nM), the composition of the invention alone (one dose 1 μg/ml), and the combination of both compounds in one glioma cell line D-54MG. FIG. 7A represents the percentage of dead cells. Each compound alone does not produce cell death in glioma cells, however the combination induces significant cell death, indicating that the composition of the invention in combination with known cytostatic agent such as rapamycin or any analogs, can produce tumor cells death. This effect depends on the Rapamycin concentration.

2-Deoxyglucose (2-DOG) is known blocker of glycolysis, in the following example we show that the combination of the invention in combination with 17 mM 2-DOG, triggered cancer cell death in a dose-dependent manner (FIG. 7B).

### Example 8

### The Composition of the Invention Blocks the Inner Mitochondrial Membrane Gradient (Δψm) in Tumor Cells

Mitochondria were isolated from tumor cells using SIGMA Mitochondria Isolation Kit. Δψm were measured in presence of the composition of the invention (1-5 μg/ml) in the isolated mitochondria by mean of uptake of the cationic carbocyanine dye JC-1 (5,5′,6,6′-tetrachloro-1,1′,3,3′-tetraethybenzene-imidazol carbocyanine iodine) into the matrix. The results demonstrate a reduction of Δψm of about 80% compared to the control. This result indicates that the composition of the invention is affecting the inner mitochondrial potential when applied directly to live mitochondria from tumor cells and therefore can be defined as a blocker of Δψm.

### Example 9

### The Composition of the Invention Acts as an Anti-Viral (Virostatic) Agent

The composition of the invention has been used to treat a 12-14 months old cat (the subject) diagnosed with Feline Leukemia virus (FeLV) through vital clinical signs as well as Elisa test for detection of FeLV.

At the time of the visit to the owner's veterinarian, the subject presented signs such as anaemia, loss of appetite, loss of body mass, respiratory distress, abdominal pain, lethargy, kidney disease. The composition of the invention CEG-002 was then administered to the subject as follows: 1.5 ml of the composition administrated orally for body weight of approximately 5 Kg, 3 times a day for 2 weeks. During the three first days of treatment, the subject recovered its appetite and was able to drink and eat. After two weeks of treatment, the subject had recovered complete activity and the treatment was stopped.

The subject was brought back to the veterinarian where clinical signs as well as virus titration confirmed complete remission. Nine months after first diagnostic, the subject was still well and active.

### REFERENCES

Greiner et al. Glucose is essential for proliferation and the glycolytic enzyme induction that provokes a transition to glycolytic energy production. J. Biol. Chem. 1994, 269, 31484-31490.
Griguer C, Oliva C, Gillespie G. Y. Glucose Metabolism Heterogeneity in Human and Mouse Malignant Glioma Cell Lines. J. Neuro-oncology, in press
Warburg O. On the origin of cancer cells. Science. 1956, 123, 309-314
Ziegler A. et al. High glycolitic activity in rat gliomas demonstrated in vivo by correlation peak 1H magnetic resonance imaging. Cancer Res. 2001, 61, 5595-5600
Hossmann K A et al. Regional metabolism of experimental brain tumors. Acta Neuropathol. 1986, 69, 139-147
Hossmann K A et al. Bioluminescence and fluoroscopic imaging of tissue pH and metabolites in experimental brain tumors of cat. NMR Biomed. 1992, 5, 259-264
Urbano A M et al. Effect of overexpression of the liver sub-unit of 6-phosphofructo-1-kinaseon the metabolism of cultured mammalian cell line. Biochem. J. 2000, 352, 921-927
Dang C V and Semenza G L. Oncogenic alterations of metabolism. Trends Biochem. Sci. 1999, 24, 68-72
Bani et al. Gene expression correlating with response to paclitaxel in ovarian carcinoma xenograft. Mol. Cancer. Ther. 2004, 3(2), 111-121
Blanquicett, C., G. Y. Gillespie, L. B. Nabors, C. R. Miller, S. Bharara, D. J. Buchsbaum, R. B. Diasio, M. R. Johnson Induction of thymidine phosphorylase in both irradiated and shielded, contralateral human U87MG glioma xenografts: Implications for a dual modality treatment using Capecitabine and irradiation. Molecular Cancer Therapeutics 2002, 1, 1139-1145
Watson M A, Perry A, Budhjara V, Hicks C, Shannon W D, Rich K M. Gene expression profiling with oligonucleotide microarrays distinguishes World Health Organization grade of oligodendrogliomas. Cancer Res. 2001.
Kim S. et al. Identification of combination gene sets for glioma classification. Molecular Cancer Therapeutics. 2002 1, 1229-1236
Pomeroy S L et al. Prediction of central nervous system embryonal tumor outcome based gene expression. Nature, 2002, 415: 436-442
MacDonald T J, Brown K M, LaFleur B, Peterson K, Lawlor C, Chen Y, Packer R J, Cogen P, Stephan D A. Expression profiling of medulloblastoma: pDGFRA and RAS/MAPK pathway as therapeutic targets for metastatic disease. Nat Genet. 2001, 29: 143-152
Pandita A, Aldape K D, Zadeh G, Guha A, James C D. Contrasting in vivo and in vitro fates of glioblastoma cell subpopulations with amplified EGFR. Genes Chromosomes Cancer. 2004 January; 39(1):29-36.
Xiao, A., H. Wu, P. P. Pandolfi, D. N. Louis, T. Van Dyke. Astrocyte inatviation of the pRb pathway predisposes mice to malignant astrocytoma development that is accelerated by PTEN mutation. Cancer Cell, 1 157-168, 2002.
Ideker T. et al. Integrated genomic and proteomic analyses of a systematically perturbed metabolic network. 2001, Science, 292, 929-934
Preiss T. et al. Homodirectional changes in transcriptome composition and mRNA translation induced by rapamycin and heat shock. 2003, Nature Struct. Biol. 10, 1039-1047
Plowman J, Waud W R, Koutsoukos A D, Rubinstein L V, Moore T D, Grever M R. (1994) Preclinical antitumor activity of temozolomide in mice: efficacy against human brain tumor xenografts and synergism with 1,3-bis(2-chloroethyl)-1-nitrosourea. Cancer Res. 15; 54 (14):3793-9.
Brand and Hermfisse (1997) Aerobic glycolysis by proliferating cells: a protective strategy against reactive oxygen species. FASEB J. 1997 11(5):388-95.
Schmidt H, Siems W, Muller M, Dumdey R, Rapoport S M. (1991) ATP-producing and consuming processes of Ehrlich mouse ascites tumor cells in proliferating and resting phases. Exp Cell Res. 194(1):122-7.
Rodriguez-Enriquez S, Torres-Marquez M E, Moreno-Sanchez R. (2000) Substrate oxidation and ATP supply in AS-30D hepatoma cells. Arch Biochem Biophys. 1; 375 (1):21-30.
Burk D, Woods M, Hunter J. (1967) On the significance of glucolysis for cancer growth, with special reference to Morris rat hepatomas. J Natl Cancer Inst. 38(6):839-63.
Hume D A, Weidemann M J. (1979) Role and regulation of glucose metabolism in proliferating cells. J Natl Cancer Inst. 62(1):3-8.
Miccoli L, Poirson-Bichat F, Sureau F, Bras Goncalves R, Bourgeois Y, Dutrillaux B, Poupon M F, Oudard S. (1998) Potentiation of lonidamine and diazepam, two agents acting on mitochondria, in human glioblastoma treatment. J Natl Cancer Inst. 16; 90(18): 1400-6.
Jones B, Dale R G. (1999) Inclusion of molecular biotherapies with radical radiotherapy: modeling of combined modality treatment schedules. Int J Radiat Oncol Biol Phys. 1; 45(4): 1025-34
Eshleman J S, Carlson B L, Mladek A C, Kastner B D, Shide K L, Sarkaria J N. (2002) Inhibition of the mammalian target of rapamycin sensitizes U87 xenografts to fractionated radiation therapy. Cancer Res. 15; 62 (24):7291-7
Delmas C, Heliez C, Cohen-Jonathan E, Bonnet J, Favre G, Toulas C. (2002) Farnesyltransferase inhibitor, R115777, reverse the resistance of human glioma cell lines to ionizing radiation. Int. J. Cancer 100: 43-48
O'Reilly S M, Newlands E S, Glaser M G, Brampton M, Rice-Edwards J M, Illingworth R D, Richards P G, Kennard C, Colquhoun I R, Lewis P, et al. (1993) Temozolomide: a new oral cytotoxic chemotherapeutic agent with promising activity against primary brain tumors. Eur J Cancer. 29A (7):940-2
Polyak, K.; Li, Y.; Zhu, H.; Lengauer, C.; Willson, J. K.; Markowitz, S. D.; Trush, M. A.; Kinzier, K. W.; Vogelstein, B. (1998) Somatic mutations of the mitochondrial genome in human colorectal tumors. Nat. Genet. 20 (3) 291-3
Yung W K, Prados M D, Yaya-Tur R, Rosenfeld S S, Brada M, Friedman H S, Albright R, Olson J, Chang S M, O'Neill A M, Friedman A H, Bruner J, Yue N, Dugan M, Zaknoen S, Levin V A. (1999) Multicenter phase II trial of temozolomide in patients with anaplastic astrocytoma or anaplastic oligoastrocytoma at first relapse. Temodal Brain Tumor Group. J Clin Oncol. 1999; 17 (9):2762-71.
Denny B J, Wheelhouse R T, Stevens M F, Tsang L L, Slack J A. (1994) NMR and molecular modeling investigation of the mechanism of activation of the antitumor drug temozolomide and its interaction with DNA. Biochemistry. 9; 33(31):9045-51
D'Atri S, Piccioni D, Castellano A, Tuorto V, Franchi A, Lu K, Christiansen N, Frankel S, Rustum Y M, Papa G. (1995) Chemosensitivity to triazene compounds and O6-alkylguanine-DNA alkyltransferase levels: studies with blasts of leukaemic patients. Ann Oncol. 6(4):389-93.
D'Atri S, Tentori L, Lacal P M, Graziani G, Pagani E, Benincasa E, Zambruno G, Bonmassar E, Jiricny J. (1998) Involvement of the mismatch repair system in temozolomide-induced apoptosis. Mol. Pharmacol.; 54(2):334-41
Joshi B, Li L, Taffe B G, Zhu Z, Wahl S, Tian H, Ben-Josef E, Taylor J D, Porter A T, Tang D G. Apoptosis induction by a novel anti-prostate cancer compound, BMD188 (a fatty acid-containing hydroxamic acid), requires the mitochondrial respiratory chain. Cancer Res. 1999 Sep. 1; 59(17):4343-55.
Li L, Zhu Z, Joshi B, Zhang C, Johnson C R, Marnett L J, Honn K V, Crissman J D, Porter A T, Tang D G. A novel hydroxamic acid compound, BMD188, demonstrates anti-prostate cancer effects by inducing apoptosis. II: In vivo efficacy and pharmacokinetic studies.
Anticancer Res. 1999 January-February; 19(1A):61-9.
Li L, Zhu Z, Joshi B, Porter A T, Tang D G. A novel hydroxamic acid compound, BMD188, demonstrates anti-prostate cancer effects by inducing apoptosis. I: In vitro studies. Anticancer Res. 1999 January-February; 19(1A):51-60.
Tang D G, Li L, Zhu Z, Joshi B, Johnson C R, Marnett L J, Honn K V, Crissman J D, Krajewski S, Reed J C, Timar J, Porter A T. BMD188, A novel hydroxamic acid compound, demonstrates potent anti-prostate cancer effects in vitro and in vivo by inducing apoptosis: requirements for mitochondria, reactive oxygen species, and proteases. Pathol Oncol Res. 1998; 4(3):179-90.
D'Souza G G, Weissig V. Approaches to mitochondrial gene therapy.
Curr Gene Ther. 2004 September; 4(3):317-28.
Zeviani M, Di Donato S. Mitochondrial disorders. Brain. 2004 October; 127(Pt 10):2153-72.
Fernie A R, Carrari F, Sweetlove L J. Respiratory metabolism: glycolysis, the TCA cycle and mitochondrial electron transport. Curr Opin Plant Biol. 2004 June; 7(3):254-61.
Dalton W S. Targeting the mitochondria: an exciting new approach to myeloma therapy. Clin Cancer Res. 2002 December; 8(12):3643-5.
Don A S, Hogg P J. Trends Mol. Med. 2004 August; 10(8):372-8. Mitochondria as cancer drug targets.
Grad J M, Cepero E, Boise L H. Drug Resist Updat. 2001 April; 4(2):85-91. Mitochondria as targets for established and novel anti-cancer agents.
Isidoro A, Martinez M, Fernandez P L, Ortega A D, Santamaria G, Chamorro M, Reed J C, Cuezva J M. Biochem J. 2004 Feb. 15; 378(Pt 1):17-20. Alteration of the bioenergetic phenotype of mitochondria is a hallmark of breast, gastric, lung and oesophageal cancer.
Modica-Napolitano J S, Singh K. Expert Rev Mol. Med. 2002 Apr. 11; 2002:1-19. Mitochondria as targets for detection and treatment of cancer.
Morgan J, Oseroff A R. Adv Drug Deliv Rev. 2001 Jul. 2; 49(1-2):71-86. Mitochondria-based photodynamic anti-cancer therapy.
Morisaki T, Katano M. Curr Med. Chem. 2003 December; 10(23):2517-21. Mitochondria-targeting therapeutic strategies for overcoming chemoresistance and progression of cancer.
Pedersen P L. Prog Exp Tumor Res. 1978; 22:190-274. Tumor mitochondria and the bioenergetics of cancer cells.

What is claimed is:

1. A synergistic composition comprising:
a) a first compound consisting of a quinone derivative of the general formula A 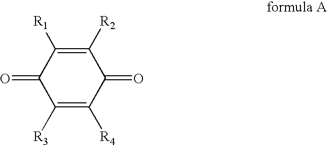
where R₁, R₂, R₃, R₄ are the same or different and are selected from the group consisting of H, OH, CH₃, OCH₃, CHO, COOH, halogen atoms (Br; Cl; F; I), NO₂, NH₂ and SO₃H;
b) a second compound selected from the group consisting of:
a 3-cyclobutene-1,2 dione derivative of the general formula B1 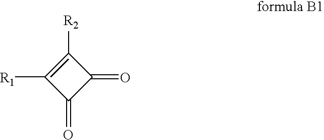
where R₁ and R₂ are the same or different and are selected from the group consisting of H, OH, CH₃, OCH₃, CHO, COOH, halogen atoms (Br; Cl; F; I), NO₂, NH₂ and SO₃H;
a 4-cyclopentene-1,2,3, trione derivative of the general formula B2 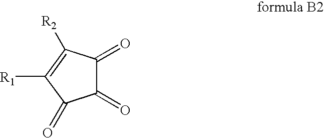
where R₁ and R₂ are the same or different and are selected from the group consisting of H, OH, CH₃, OCH₃, CHO, COOH, halogen atoms (Br; Cl; F; I), NO₂, NH₂ and SO₃H; and
a 5-cyclohexene-1,2,3,4 tetraone derivative of the general formula B3 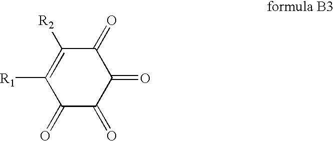
where R₁ and R² are the same or different and are chosen from the group consisting of H, OH, CH₃, OCH₃, CHO, COOH, halogen atoms (Br; Cl; F; I), NO₂, NH₂ and SO₃H; and
c) a third compound consisting of a benzene derivative of the general formula C 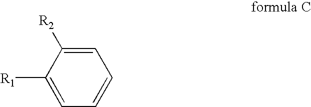
where R₁ and R₂ are the same or different and are chosen from the group consisting of H, OH, CH₃, OCH₃, CHO, COOH, halogen atoms (Br; Cl; F; I), NO₂, NH₂ and SO₃H.

2. The synergistic composition as defined in claim 1, wherein said synergistic composition is selected from the group consisting of a cytostatic agent, an antibiotic agent and a virostatic agent.

3. The synergistic composition as defined in claim 2, wherein said cytostatic agent is a cytostatic agent against cancer cells.

4. The synergistic composition as defined in claim 1, wherein the first compound is a tetrahydroxy-p-benzoquinone, the second compound is selected from the group consisting of squaric acid, croconic acid and 5,6-dihydroxy-5-cyclohexen-1,2,3,4-tetraone and the third compound is catechol.

5. The synergistic composition as defined in claim 4, wherein said synergistic composition is used to treat tumor cells in a subject.

6. The synergistic composition as defined in claim 4, wherein said synergistic composition is selected from the group consisting of a cytostatic agent, an antibiotic agent and a virostatic agent.

7. The synergistic composition as defined in claim 6, wherein said cytostatic agent is a cytostatic agent against cancer cells.

8. The synergistic composition as defined in claim 6, wherein said cytotstatic agent is a cytostatic agent against a glioma or an adenocarcinoma.

9. A pharmaceutical composition comprising:
the synergistic composition as defined in claim 1; and
a pharmaceutically acceptable carrier.

10. A pharmaceutical composition comprising:
the synergistic composition as defined in claim 2; and
a pharmaceutically acceptable carrier.

11. A method for treatment of cancer, comprising the step of administering to a subject in need thereof, an effective amount of the synergistic composition as defined in claim 1.

12. The method for treatment of cancer according to claim 11, further comprising administering radiation therapy to said subject.

13. The method for treatment of cancer according to claim 11, further comprising administering a chemotherapeutic agent to said subject.

14. A method for treatment of cancer, comprising the step of administering to a subject in need thereof, an effective amount of the synergistic composition as defined in claim 4.

15. A method of inducing cytostasis in a cell, comprising administering to said cell an effective amount of the composition as defined in claim 1.

16. The method of inducing cytostasis in a cell as defined in claim 15, wherein said cell is a cancer cell.

17. A method for treating a bacterial infection in a subject in need thereof, comprising the step of administering to said subject, an effective amount of the synergistic composition as defined in claim 1.

18. A method for treating a bacterial infection in a subject in need thereof, comprising the step of administering to said subject, an effective amount of the synergistic composition as defined in claim 4.

19. A method for treating a viral infection in a subject in need thereof, comprising the step of administering to said subject, an effective amount of a composition as defined in claim 1.

20. A method for treating a viral infection in a subject in need thereof, comprising the step of administering to said subject, an effective amount of a composition as defined in claim 4.
